(19) United States
(12) Patent Application Publication
(10) Pub. No.: US 2025/0369124 A1
(43) Pub. Date: Dec. 4, 2025

(54) IMIDAZOLINE-SUGAR CONJUGATE COMPOSITIONS AND METHODS FOR INHIBITING CORROSION

(71) Applicant: Ecolab USA Inc., St. Paul, MN (US)

(72) Inventors: Jonathan MASERE, Richmond, TX (US); Ashish DHAWAN, Aurora, IL (US); Kameswara VYAKARANAM, Sugar Land, TX (US)

(73) Assignee: Ecolab USA Inc., St. Paul, MN (US)

(21) Appl. No.: 19/221,042

(22) Filed: May 28, 2025

Related U.S. Application Data
(60) Provisional application No. 63/654,923, filed on May 31, 2024.

(51) Int. Cl. C23F 11/14; C07D 233/16; C07H 17/02

(52) U.S. Cl. CPC C23F 11/149; C07D 233/16; C07H 17/02

(57) ABSTRACT

Corrosion inhibitor compositions that include a compound of formula (I) are provided. In general, the compound of formula (I) is an environmentally benign sugar-derivatized imidazoline having a lipophilic group. A method of reducing corrosion of a metal surface in contact with an aqueous system that includes adding the disclosed corrosion inhibitor composition to the aqueous system is also provided. Also provided are processes for preparing corrosion inhibitor compounds of formula (I).

### TECHNICAL FIELD

The present disclosure generally relates to corrosion inhibitor compositions and methods of inhibiting corrosion using the same. More particularly, the disclosure pertains to compositions including sugar-derivatized imidazolines and methods of inhibiting corrosion using the same.

### BACKGROUND

Equipment and infrastructure used in the recovery, transportation storage, and processing of oil and natural gas come into contact with highly corrosive agents. These corrosive agents include brines, organic acids, carbon dioxide and hydrogen sulfide. In addition to exposure to these highlight corrosive agents, damage to the equipment and infrastructure is frequently exacerbated by erosion from insoluble materials including sand and other inorganic solids.

Processing equipment is particularly vulnerable to damage from corrosive agents given the metallurgical properties of the materials used to fabricate this equipment. Common metals used in processing equipment that are particularly vulnerable to damage from corrosion and erosion include metallic chrome steel compositions, ferritic alloy steel compositions, austenitic steel compositions, precipitation-hardened steel compositions, and high nickel steel compositions. In particular, carbon steel compositions are frequently used in the fabrication of processing equipment, given its comparatively low cost.

The high vulnerability of the materials used to fabricate oil and gas equipment to corrosion makes the use of corrosion inhibitors a necessity. Unfortunately, a major constraint in this field is the limited selection of environmentally benign corrosion inhibitors. Waste streams containing these corrosion inhibitors are liable to end up in the environment, for example, through accidental leakage or routine disposal. Of the most commonly used corrosion inhibitors, few are derived from sustainable sources and the vast majority are not environmentally benign. Consequently, there is a significant unmet need for environmentally benign corrosion inhibitors for protecting equipment and infrastructure used in the production, transportation, storage, and processing of oil and gas. The discovery and implementation of such corrosion inhibitors is essential for maintaining equipment and infrastructure integrity with minimal damage to the environment.

To be effective, corrosion inhibitors, must typically have long lipophilic hydrocarbon moieties and compact polar functional head groups at one end of the lipophilic chain. Without being bound to theory, corrosion inhibitors of this type function as surface-active compounds, where the polar head groups bind to the metal surface while the non-polar tails protrude away from the metal surface. The resultant hydrophobic films formed by the hydrophobic tails protruding from the metal surface protect the metal surfaces from water-borne corrosive agents.

One method for improving the effectiveness of corrosion inhibitors is to increase the overall polarity of the polar head groups. Amphiphilic quaternary ammonium compounds have been developed and employed as effective corrosion inhibitors, but many are not derived from sustainable sources and are, likewise, not biodegradable or environmentally benign. Thus, there is a significant need to develop environmentally benign and biodegradable corrosion inhibitors that are derived from sustainable sources.

The present disclosure addresses this need by describing novel corrosion inhibitor compounds possessing naturally-derived sugar substituents that enhance the polarity of the head groups, thereby improving the efficacy of the corrosion inhibitors. Moreover, because the sugar substituents are derived from natural, sustainable sources, the compounds disclosed herein have enhanced biodegradability and are, therefore, environmentally benign. Thus, the present disclosure addresses a significant unmet need by providing effective, environmentally benign corrosion inhibitors for use in a variety of applications, including the oil and natural gas industries.

### BRIEF SUMMARY

Provided herein are corrosion inhibitor compositions that include a compound of formula (I):

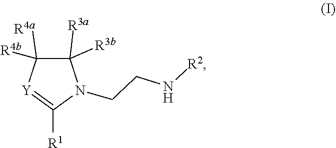

or a salt thereof, wherein:
Y is N or N⁺—R⁵;
R¹ is an optionally substituted C₈-C₂₀ alkyl, an optionally substituted C₈-C₂₀ alkenyl, an optionally substituted C₈-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl);
R² is a carbohydrate group;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R⁵ is H, optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl).

In some embodiments, the corrosion inhibitor composition includes a compound of formula (I-A), (I-B), or (I-C):

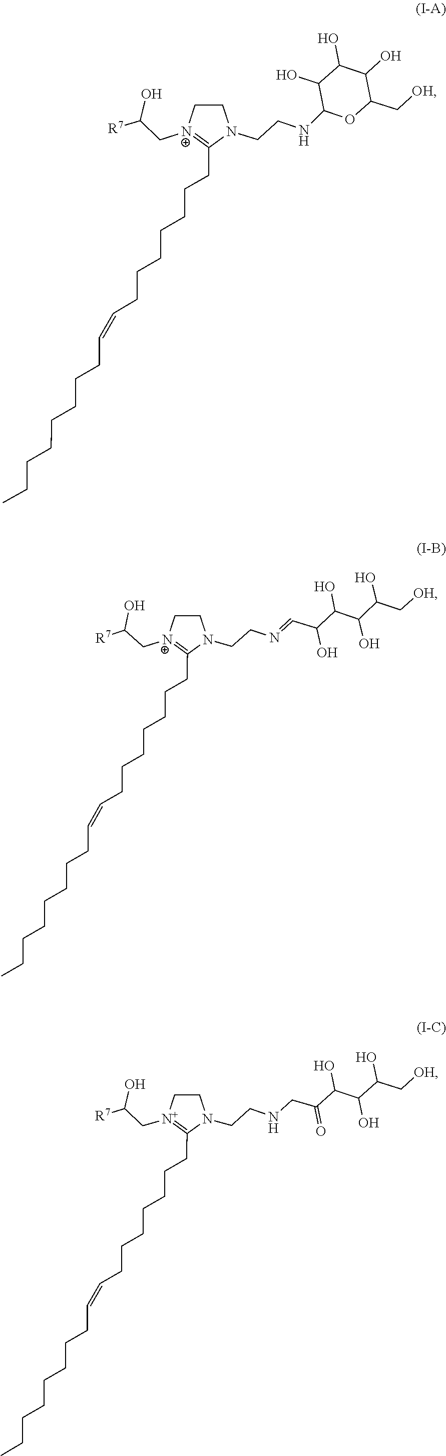

or a salt thereof, wherein:
R⁷ is H, optionally substituted C₁-C₂₀ alkyl, optionally substituted
—(C₁-C₂₀ alkylene)-(C₆-C₂₀ aryl), or optionally substituted
—(C₁-C₂₀ alkylene)-(C₆-C₂₀ aryl)-(C₁-C₂₀ alkyl).

A method of reducing corrosion of a metal surface in contact with an aqueous system is also provided, where the method includes adding a corrosion inhibitor composition of a compound of formula (I) to the aqueous system. The aqueous system may include one or more corrosive agents, including, but not limited to, brine, organic acids, carbon dioxide, and hydrogen sulfide. The metal surface of the aqueous system may include metallic-chrome steel, ferritic-alloy steel, austenitic-steel, precipitation-hardened steel, high-nickel steel, carbon steel, or a combination thereof.

Also provided is a process for preparing a compound of formula (I) that includes contacting a compound of formula (II):

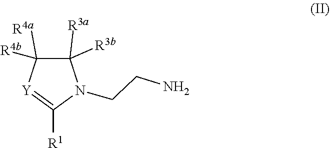

with a carbohydrate, wherein Y, R¹, R², R^{3a}, R^{3b}, R^{4a}, R^{4b}, and R⁵ are as described above.

The foregoing has outlined rather broadly the features and technical advantages of the present disclosure in order that the detailed description that follows may be better understood. Additional features and advantages of the disclosure will be described hereinafter that form the subject of the claims of this application.

### DETAILED DESCRIPTION

Various embodiments are described below. The relationship and functioning of the various elements of the embodiments may better be understood by reference to the following detailed description. However, embodiments are not strictly limited to those described below.

Examples of methods and materials are described below, although methods and materials similar or equivalent to those described herein can be used in practice or testing of the present disclosure. All publications, patent applications, patents and other reference materials mentioned herein are incorporated by reference in their entirety. The materials, methods, and examples disclosed herein are illustrative only and not intended to be limiting.

Unless otherwise defined, all technical and scientific terms used herein have the same meaning as commonly understood by one of ordinary skill in the art. In case of conflict, the present document, including definitions, will control.

Unless otherwise indicated, an alkyl group as described herein alone or as part of another group is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

Any alkyl functional group disclosed herein may be derived from, for example, an animal fatty acid or a vegetable oil fatty acid, such as a soy fatty acid, a tall oil fatty acid, a canola oil fatty acid, oleic acid, glycerol-restricted avocado oil fatty acid, corn oil fatty acid, cottonseed oil fatty acid, grape seed oil fatty acid, hazelnut oil fatty acid, hemp seed oil fatty acid, linseed oil fatty acid, olive oil fatty acid, palm kernel oil fatty acid, peanut seed oil fatty acid, rape seed oil fatty acid, rice bran oil fatty acid, safflower oil fatty acid, sesame oil fatty acid, soybean oil fatty acid, sunflower seed oil fatty acid, and walnut oil fatty acid. In some instances, a glycerol-restricted vegetable oil fatty acid may comprise, consist of, or consist essentially of glycerol-restricted soybean oil fatty acid.

“Alkenyl” refers to a straight or branched hydrocarbon having, for example, 2, 3, 4, 5, 6, 7, 8, 9, 10, 11, 12, 13, 14, 15, or 16 carbon atoms, and having one or more carbon-carbon double bonds. Alkenyl groups include, but are not limited to, ethenyl, 1-propenyl, 2-propenyl (allyl), iso-propenyl, 2-methyl-1-propenyl, 1-butenyl, and 2-butenyl. Alkenyl groups may be unsubstituted or substituted by one or more suitable substituents.

“Alkynyl” refers to a straight or branched hydrocarbon having, for example, 2, 3, 4, 5, 6, 7, 8, 9, 10, 11, 12, 13, 14, 15, or 16 carbon atoms, and having one or more carbon-carbon triple bonds. Alkynyl groups include, but are not limited to, ethynyl, propynyl, and butynyl. Alkynyl groups may be unsubstituted or substituted by one or more suitable substituents.

“Halogen” or “halo” refers to F, Cl, Br, and I.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C═O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

“Aqueous system” refers to any system containing one or more metallic surfaces/components, which are in contact with an aqueous medium (e.g., water) on a periodic or continuous basis.

“Industrial water system” means any system that circulates water as a component. Non-limiting examples of “industrial water systems” include cooling systems, boiler systems, heating systems, membrane systems, paper making systems, food and beverage systems, oil and gas systems, and any other system that circulates or includes water.

The present disclosure relates to corrosion inhibitor compositions, methods of inhibiting corrosion, and formulations useful for inhibiting corrosion. Inhibiting corrosion includes, for example, reducing corrosion, completely eliminating corrosion or prohibiting corrosion from occurring for some period of time, lowering a rate of corrosion, etc. In some aspects, the corrosion inhibitor compositions are useful for inhibiting corrosion of metallic surfaces in aqueous environments. In some aspects, the corrosion inhibitor compositions and/or formulations comprise one or more sugar-derivatized imidazolines having a lipophilic tail group.

A corrosion inhibitor composition comprising a compound of formula (I):

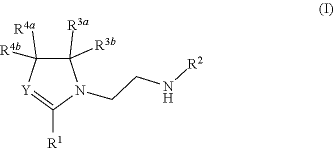

or a salt thereof, is provided, wherein:
Y is N or N⁺—R⁵;
R¹ is an optionally substituted C₅-C₂₀ alkyl, an optionally substituted C₈-C₂₀ alkenyl, an optionally substituted C₅-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl);
R² is a carbohydrate group;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R⁵ is H, optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl).

In some embodiments, Y is N.

In some embodiments, Y is N⁺—R⁵.

In some embodiments, R¹ is an optionally substituted C₈-C₂₀ alkyl, an optionally substituted C₈-C₂₀ alkenyl, an optionally substituted C₈-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl). In other embodiments, R¹ is an optionally substituted C₈-C₂₀ alkyl. In other embodiments, R¹ is C₈-C₂₀ alkyl. In other embodiments, R¹ is an unsubstituted C₈-C₂₀ alkyl. In other embodiments, R¹ is an optionally substituted C₈-C₂₀ alkenyl. In other embodiments, R¹ is C₈-C₂₀ alkenyl. In other embodiments, R¹ is an unsubstituted C₈-C₂₀ alkenyl. In other embodiments, R¹ is an optionally substituted C₈-C₂₀ alkynyl. In other embodiments, R¹ is C₈-C₂₀ alkynyl. In other embodiments, R¹ is an unsubstituted C₈-C₂₀ alkynyl. In other embodiments, R¹ is an optionally substituted —(C₆-C₂₀ aryl)-(C₄-C₂₀ alkyl). In other embodiments, R¹ is —(C₆-C₂₀ aryl)-(C₄-C₂₀ alkyl). In other embodiments, R¹ is an unsubstituted —(C₆-C₂₀ aryl)-(C₄-C₂₀ alkyl). In other embodiments, R¹ is an optionally substituted —(C₄-C₂₀ alkylene)-(C₆-C₂₀ aryl). In other embodiments, R¹ is —(C₄-C₂₀ alkylene)-(C₆-C₂₀ aryl). In other embodiments, R¹ is an unsubstituted —(C₄-C₂₀ alkylene)-(C₆-C₂₀ aryl). In other embodiments, R¹ is an optionally substituted —(C₄-C₂₀ alkylene)-(C₆-C₂₀ aryl)-(C₄-C₂₀ alkyl). In other embodiments, R¹ is —(C₄-C₂₀ alkylene)-(C₆-C₂₀ aryl)-(C₄-C₂₀ alkyl). In other embodiments, R¹ is an unsubstituted —(C₄-C₂₀ alkylene)-(C₆-C₂₀ aryl)-(C₄-C₂₀ alkyl).

In some embodiments, R¹ is an optionally substituted C₈-C₂₀ alkyl, an optionally substituted C₈-C₂₀ alkenyl, an optionally substituted C₈-C₂₀ alkynyl, an optionally substituted —(C₆-C₁₀ aryl)-(C₄-C₂₀ alkyl), an optionally substituted —(C₄-C₂₀ alkylene)-(C₆-C₁₀ aryl), or an optionally substituted —(C₄-C₂₀ alkylene)-(C₆-C₁₀ aryl)-(C₄-C₂₀ alkyl). In other embodiments, R¹ is an optionally substituted —(C₆-C₁₀ aryl)-(C₄-C₂₀ alkyl). In other embodiments, R¹ is —(C₆-C₁₀ aryl)-(C₄-C₂₀ alkyl). In other embodiments, R¹ is an unsubstituted —(C₆-C₁₀ aryl)-(C₄-C₂₀ alkyl). In other embodiments, R¹ is an optionally substituted —(C₄-C₂₀ alkylene)-(C₆-C₁₀ aryl). In other embodiments, R¹ is —(C₄-C₂₀ alkylene)-(C₆-C₁₀ aryl). In other embodiments, R¹ is an unsubstituted —(C₄-C₂₀ alkylene)-(C₆-C₁₀ aryl). In other embodiments, R¹ is an optionally substituted —(C₄-C₂₀ alkylene)-(C₆-C₁₀ aryl)-(C₄-C₂₀ alkyl). In other embodiments, R¹ is —(C₄-C₂₀ alkylene)-(C₆-C₁₀ aryl)-(C₄-C₂₀ alkyl). In other embodiments, R¹ is an unsubstituted —(C₄-C₂₀ alkylene)-(C₆-C₁₀ aryl)-(C₄-C₂₀ alkyl).

In some embodiments, R¹ is selected from octyl, octanyl, octynyl, nonyl, nonenyl, nonynyl, decyl, decenyl, decynyl, undecyl, undecenyl, undecynyl, dodecyl, dodecenyl, dodecynyl, tridecyl, tridecenyl, tridecynyl, tetradecyl, tetradecenyl, tetradecynyl, pentadecyl, pentadecenyl, pentadecynyl, hexadecyl, hexadecenyl, hexadecynyl, heptadecyl, heptadecenyl, heptadecynyl, octadecyl, octadecenyl, octadecynyl, nonadecyl, nonadecenyl, nonadecynyl, icosyl, icosenyl, and icosynyl. In other embodiments, R¹ is octyl. In other embodiments, R¹ is octanyl. In other embodiments, R¹ is octynyl. In other embodiments, R¹ is nonyl. In other embodiments, R¹ is nonenyl. In other embodiments, R¹ is nonynyl. In other embodiments, R¹ is decyl. In other embodiments, R¹ is decenyl. In other embodiments, R¹ is decynyl. In other embodiments, R¹ is undecyl. In other embodiments, R¹ is undecenyl. In other embodiments, R¹ is undecynyl. In other embodiments, R¹ is dodecyl. In other embodiments, R¹ is dodecenyl. In other embodiments, R¹ is dodecynyl. In other embodiments, R¹ is tridecyl. In other embodiments, R¹ is tridecenyl. In other embodiments, R¹ is tridecynyl. In other embodiments, R¹ is tetradecyl. In other embodiments, R¹ is tetradecenyl. In other embodiments, R¹ is tetradecynyl. In other embodiments, R¹ is pentadecyl. In other embodiments, R¹ is pentadecenyl. In other embodiments, R¹ is pentadecynyl. In other embodiments, R¹ is hexadecyl. In other embodiments, R¹ is hexadecenyl. In other embodiments, R¹ is hexadecynyl. In other embodiments, R¹ is heptadecyl. In other embodiments, R¹ is heptadecenyl. In other embodiments, R¹ is heptadecynyl. In other embodiments, R¹ is octadecyl. In other embodiments, R¹ is octadecenyl. In other embodiments, R¹ is octadecynyl. In other embodiments, R¹ is nonadecyl. In other embodiments, R¹ is nonadecenyl. In other embodiments, R¹ is nonadecynyl. In other embodiments, R¹ is icosyl. In other embodiments, R¹ is icosenyl. In other embodiments, R¹ is icosynyl

In certain embodiments, R¹ is heptadecenyl. In certain other embodiments, R¹ is heptadec-8-enyl. In yet other embodiments, R¹ is heptadec-8-en-1-yl.

In some embodiments, R^{3a} is H, —OH, —NH₂, —NH(C₁-C₆ alkyl), —N(C₁-C₆ alkyl)₂, —CN, halo, or optionally substituted C₁-C₆ alkyl. In other embodiments, R^{3a} is H. In other embodiments, R^{3a} is —OH. In other embodiments, R^{3a} is —NH₂. In other embodiments, R^{3a} is —NH(C₁-C₆ alkyl). In other embodiments, R^{3a} is —N(C₁-C₆ alkyl)₂. In other embodiments, R^{3a} is —CN. In other embodiments, R^{3a} is halo. In other embodiments, R^{3a} is optionally substituted C₁-C₆ alkyl. In other embodiments, R^{3a} is C₁-C₆ alkyl. In other embodiments, R^{3a} is unsubstituted C₁-C₆ alkyl.

In some embodiments, R^{3b} is H, —OH, —NH₂, —NH(C₁-C₆ alkyl), —N(C₁-C₆ alkyl)₂, —CN, halo, or optionally substituted C₁-C₆ alkyl. In other embodiments, R^{3b} is H. In other embodiments, R^{3b} is —OH. In other embodiments, R^{3b} is —NH₂. In other embodiments, R^{3b} is —NH(C₁-C₆ alkyl). In other embodiments, R^{3b} is —N(C₁-C₆ alkyl)₂. In other embodiments, R^{3b} is —CN. In other embodiments, R^{3b} is halo. In other embodiments, R^{3b} is optionally substituted C₁-C₆ alkyl. In other embodiments, R^{3b} is C₁-C₆ alkyl. In other embodiments, R^{3b} is unsubstituted C₁-C₆ alkyl.

In some embodiments, R^{4a} is H, —OH, —NH₂, —NH(C₁-C₆ alkyl), —N(C₁-C₆ alkyl)₂, —CN, halo, or optionally substituted C₁-C₆ alkyl. In other embodiments, R^{4a} is H. In other embodiments, R^{4a} is —OH. In other embodiments, R^{4a} is —NH₂. In other embodiments, R^{4a} is —NH(C₁-C₆ alkyl). In other embodiments, R^{4a} is —N(C₁-C₆ alkyl)₂. In other embodiments, R^{4a} is —CN. In other embodiments, R^{4a} is halo. In other embodiments, R^{4a} is optionally substituted C₁-C₆ alkyl. In other embodiments, R^{4a} is C₁-C₆ alkyl. In other embodiments, R^{4a} is unsubstituted C₁-C₆ alkyl.

In some embodiments, R^{4b} is H, —OH, —NH₂, —NH(C₁-C₆ alkyl), —N(C₁-C₆ alkyl)₂, —CN, halo, or optionally substituted C₁-C₆ alkyl. In other embodiments, R^{4b} is H. In other embodiments, R^{4b} is —OH. In other embodiments, R^{4b} is —NH₂. In other embodiments, R^{4b} is —NH(C₁-C₆ alkyl). In other embodiments, R^{4b} is —N(C₁-C₆ alkyl)₂. In other embodiments, R^{4b} is —CN. In other embodiments, R^{4b} is halo. In other embodiments, R^{4b} is optionally substituted C₁-C₆ alkyl. In other embodiments, R^{4b} is C₁-C₆ alkyl. In other embodiments, R^{4b} is unsubstituted C₁-C₆ alkyl.

In some embodiments, R⁵ is H, optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl). In other embodiments, R⁵ is H. In other embodiments, R⁵ is optionally substituted C₁-C₂₀ alkyl. In other embodiments, R⁵ is C₁-C₂₀ alkyl. In other embodiments, R⁵ is unsubstituted C₁-C₂₀ alkyl. In other embodiments, R⁵ is optionally substituted C₂-C₂₀ hydroxyalkyl. In other embodiments, R⁵ is optionally substituted C₂-C₂₀ β-hydroxyalkyl. In other embodiments, R⁵ is C₂-C₂₀ hydroxyalkyl. In other embodiments, R⁵ is C₂-C₂₀ β-hydroxyalkyl. In other embodiments, R⁵ is unsubstituted C₂-C₂₀ hydroxyalkyl. In other embodiments, R⁵ is unsubstituted C₂-C₂₀ β-hydroxyalkyl. In other embodiments, R⁵ is optionally substituted —(C₁-C₂₀ alkylene)-(C₆-C₂₀ aryl). In other embodiments, R⁵ is —(C₁-C₂₀ alkylene)-(C₆-C₂₀ aryl). In other embodiments, R⁵ is unsubstituted —(C₁-C₂₀ alkylene)-(C₆-C₂₀ aryl). In other embodiments, R⁵ is optionally substituted —(C₂-C₂₀ hydroxyalkylene)-(C₆-C₂₀ aryl). In other embodiments, R⁵ is optionally substituted —(C₂-C₂₀ β-hydroxyalkylene)-(C₆-C₂₀ aryl). In other embodiments, R⁵ is —(C₂-C₂₀ hydroxyalkylene)-(C₆-C₂₀ aryl). In other embodiments, R⁵ is-(C₂-C₂₀ β-hydroxyalkylene)-(C₆-C₂₀ aryl). In other embodiments, R⁵ is unsubstituted —(C₂-C₂₀ hydroxyalkylene)-(C₆-C₂₀ aryl). In other embodiments, R⁵ is unsubstituted —(C₂-C₂₀ β-hydroxyalkylene)-(C₆-C₂₀ aryl). In other embodiments, R⁵ is optionally substituted —(C₂-C₂₀ hydroxyalkylene)-(C₆-C₂₀ aryl)-(C₆-C₂₀ alkyl). In other embodiments, R⁵ is optionally substituted —(C₂-C₂₀ β-hydroxyalkylene)-(C₆-C₂₀ aryl)-(C₆-C₂₀ alkyl). In other embodiments, R⁵ is —(C₂-C₂₀ hydroxyalkylene)-(C₆-C₂₀ aryl)-(C₆-C₂₀ alkyl). In other embodiments, R⁵ is —(C₂-C₂₀ β-hydroxyalkylene)-(C₆-C₂₀ aryl)-(C₆-C₂₀ alkyl). In other embodiments, R⁵ is unsubstituted —(C₂-C₂₀ hydroxyalkylene)-(C₆-C₂₀ aryl)-(C₆-C₂₀ alkyl). In other embodiments, R⁵ is unsubstituted —(C₂-C₂₀ β-hydroxyalkylene)-(C₆-C₂₀ aryl)-(C₆-C₂₀ alkyl).

In some embodiments, R⁵ is H, optionally substituted C₁-C₂₀ alkyl, optionally substituted C₂-C₂₀ hydroxyalkyl, optionally substituted —(C₁-C₂₀ alkylene)-(C₆-C₁₀ aryl), optionally substituted —(C₂-C₂₀ hydroxyalkylene)-(C₆-C₁₀ aryl), or optionally substituted —(C₂-C₂₀ hydroxyalkylene)-(C₆-C₁₀ aryl)-(C₆-C₂₀ alkyl). In other embodiments, R⁵ is optionally substituted —(C₁-C₂₀ alkylene)-(C₆-C₁₀ aryl). In other embodiments, R⁵ is —(C₁-C₂₀ alkylene)-(C₆-C₁₀ aryl). In other embodiments, R⁵ is unsubstituted —(C₁-C₂₀ alkylene)-(C₆-C₁₀ aryl). In other embodiments, R⁵ is optionally substituted —(C₂-C₂₀ hydroxyalkylene)-(C₆-C₁₀ aryl). In other embodiments, R⁵ is —(C₂-C₂₀ hydroxyalkylene)-(C₆-C₁₀ aryl). In other embodiments, R⁵ is unsubstituted —(C₂-C₂₀ hydroxyalkylene)-(C₆-C₁₀ aryl). In other embodiments, R⁵ is optionally substituted —(C₂-C₂₀ hydroxyalkylene)-(C₆-C₁₀ aryl)-(C₆-C₂₀ alkyl). In other embodiments, R⁵ is —(C₂-C₂₀ hydroxyalkylene)-(C₆-C₁₀ aryl)-(C₆-C₂₀ alkyl). In other embodiments, R⁵ is unsubstituted —(C₂-C₂₀ hydroxyalkylene)-(C₆-C₁₀ aryl)-(C₆-C₂₀ alkyl).

In some embodiments, R⁵ is optionally substituted —CH₂CH(OH)-phenyl. In some embodiments, R⁵ is —CH₂CH(OH)-phenyl. In some embodiments, R⁵ is unsubstituted —CH₂CH(OH)-phenyl.

In some embodiments, R² is a carbohydrate group. In some embodiments, R² is a reducing sugar group. The terms “reducing sugar” and “reducing sugar group”, as used herein, refers to any sugar that is capable of acting as a reducing agent. For example, in some instances, bulk reducing sugars contain some portion thereof that exists as an aldehyde or ketone, allowing the sugar to act as a reducing agent. Reducing sugars may be monosaccharides, disaccharides, oligosaccharides, or polysaccharides in some instances. Reducing sugars may be further divided into aldoses, having an aldehyde group, and ketoses, having a ketone group.

In some embodiments, R² is a reducing sugar, wherein the reducing sugar is an aldose. In some embodiments, R² is a reducing sugar, wherein the reducing sugar is a ketose.

In some embodiments, R² is a reducing monosaccharide or a reducing disaccharide. In other embodiments, R² is a reducing monosaccharide. In other embodiments, R² is a reducing disaccharide.

In some embodiments, R² is selected from glyceraldehyde, erythrose, threose, arabinose, lyxose, ribose, xylose, allose, altrose, glucose, mannose, gulose, idose, galactose, talose, fructose, sedoheptulose, mannoheptulose, lactose, maltose, and sucrose. In other embodiments, R² is glyceraldehyde. In other embodiments, R² is erythrose. In other embodiments, R² is threose. In other embodiments, R² is arabinose. In other embodiments, R² is lyxose. In other embodiments, R² is ribose. In other embodiments, R² is xylose. In other embodiments, R² is allose. In other embodiments, R² is altrose. In other embodiments, R² is glucose. In other embodiments, R² is mannose. In other embodiments, R² is gulose. In other embodiments, R² is idose. In other embodiments, R² is galactose. In other embodiments, R² is talose. In other embodiments, R² is fructose. In other embodiments, R² is sedoheptulose. In other embodiments, R² is mannoheptulose. In other embodiments, R² is lactose. In other embodiments, R² is maltose. In other embodiments, R² is sucrose.

In some embodiments, R² is a reducing sugar group, wherein the sugar is attached to formula (I) as a Maillard adduct. In some embodiments, R² is a Maillard adduct of a reducing sugar. One of ordinary skill in the art will appreciate that the Maillard reaction refers to the reaction of the carbonyl of a reducing sugar with a nucleophilic amine group to form a Schiff base. In some instances, the Schiff base may undergo further reactions or rearrangements. By way of non-limiting example, a Maillard adduct may form by the reaction of an optionally substituted imidazoline having a terminal primary amine and a reducing hexose, as illustrated below:

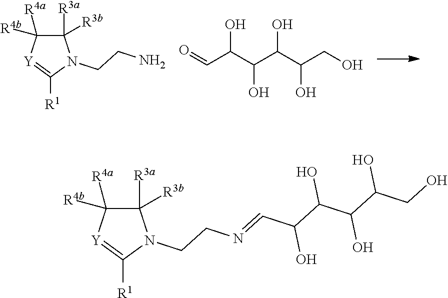

In some embodiments, R² is a reducing sugar, wherein the sugar is attached to formula (I) as an Amadori adduct. In some embodiments, R² is an Amadori adduct of a reducing sugar. One of ordinary skill in the art will appreciate that the Amadori adduct is the product of a rearrangement reaction of an N-glycoside, as may be formed by the Maillard reaction, for example. By way of non-limiting example, an Amadori adduct may form by the rearrangement of the Maillard adduct described above, as illustrated below:

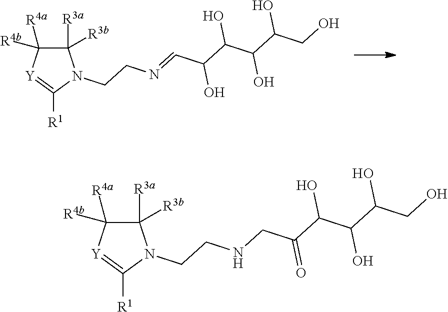

In some embodiments, the compound of formula (I) is a compound of formula (I-A), (I-B), or (I-C):

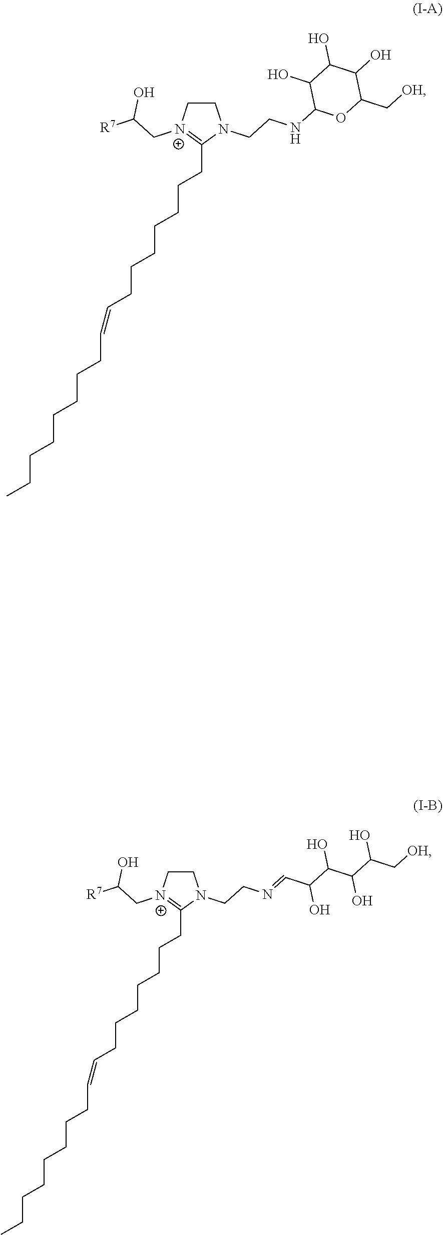

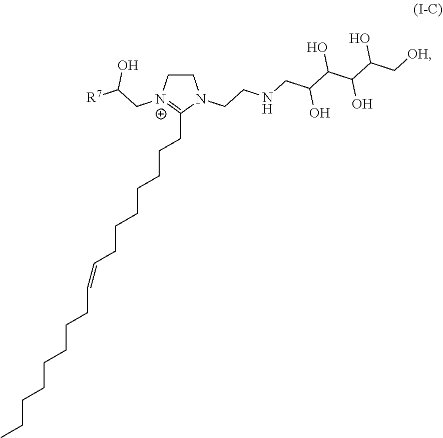

or a salt thereof, wherein:
R⁷ is H, optionally substituted C₁-C₂₀ alkyl, optionally substituted C₆-C₂₀ aryl, optionally substituted —(C₁-C₂₀ alkylene)-(C₆-C₂₀ aryl), or optionally substituted —(C₁-C₂₀ alkylene)-(C₆-C₂₀ aryl)-(C₁-C₂₀ alkyl).

In some embodiments, the compound of formula (I) is a compound of formula (I-A), wherein R⁷ is H. In some embodiments, the compound of formula (I) is a compound of formula (I-A), wherein R⁷ is optionally substituted C₁-C₂₀ alkyl. In some embodiments, the compound of formula (I) is a compound of formula (I-A), wherein R⁷ is C₁-C₂₀ alkyl. In some embodiments, the compound of formula (I) is a compound of formula (I-A), wherein R⁷ is unsubstituted C₁-C₂₀ alkyl. In some embodiments, the compound of formula (I) is a compound of formula (I-A), wherein R⁷ is optionally substituted C₆-C₂₀ aryl. In some embodiments, the compound of formula (I) is a compound of formula (I-A), wherein R⁷ is C₆-C₂₀ aryl. In some embodiments, the compound of formula (I) is a compound of formula (I-A), wherein R⁷ is unsubstituted C₆-C₂₀ aryl. In some embodiments, the compound of formula (I) is a compound of formula (I-A), wherein R⁷ is optionally substituted —(C₁-C₂₀ alkylene)-(C₆-C₂₀ aryl). In some embodiments, the compound of formula (I) is a compound of formula (I-A), wherein R⁷ is —(C₁-C₂₀ alkylene)-(C₆-C₂₀ aryl). In some embodiments, the compound of formula (I) is a compound of formula (I-A), wherein R⁷ is unsubstituted —(C₁-C₂₀ alkylene)-(C₆-C₂₀ aryl). In some embodiments, the compound of formula (I) is a compound of formula (I-A), wherein R⁷ is optionally substituted —(C₁-C₂₀ alkylene)-(C₆-C₂₀ aryl)-(C₁-C₂₀ alkyl). In some embodiments, the compound of formula (I) is a compound of formula (I-A), wherein R⁷ is —(C₁-C₂₀ alkylene)-(C₆-C₂₀ aryl)-(C₁-C₂₀ alkyl). In some embodiments, the compound of formula (I) is a compound of formula (I-A), wherein R⁷ is unsubstituted —(C₁-C₂₀ alkylene)-(C₆-C₂₀ aryl)-(C₁-C₂₀ alkyl).

In some embodiments, the compound of formula (I) is a compound of formula (I-A), wherein R⁷ is optionally substituted phenyl. In some embodiments, the compound of formula (I) is a compound of formula (I-A), wherein R⁷ is phenyl. In some embodiments, the compound of formula (I) is a compound of formula (I-A), wherein R⁷ is unsubstituted phenyl.

In some embodiments, the compound of formula (I) is a compound of formula (I-B), wherein R⁷ is H. In some embodiments, the compound of formula (I) is a compound of formula (I-B), wherein R⁷ is optionally substituted C₁-C₂₀ alkyl. In some embodiments, the compound of formula (I) is a compound of formula (I-B), wherein R⁷ is C₁-C₂₀ alkyl. In some embodiments, the compound of formula (I) is a compound of formula (I-B), wherein R⁷ is unsubstituted C₁-C₂₀ alkyl. In some embodiments, the compound of formula (I) is a compound of formula (I-B), wherein R⁷ is optionally substituted C₆-C₂₀ aryl. In some embodiments, the compound of formula (I) is a compound of formula (I-B), wherein R⁷ is C₆-C₂₀ aryl. In some embodiments, the compound of formula (I) is a compound of formula (I-B), wherein R⁷ is unsubstituted C₆-C₂₀ aryl. In some embodiments, the compound of formula (I) is a compound of formula (I-B), wherein R⁷ is optionally substituted —(C₁-C₂₀ alkylene)-(C₆-C₂₀ aryl). In some embodiments, the compound of formula (I) is a compound of formula (I-B), wherein R⁷ is —(C₁-C₂₀ alkylene)-(C₆-C₂₀ aryl). In some embodiments, the compound of formula (I) is a compound of formula (I-B), wherein R⁷ is unsubstituted —(C₁-C₂₀ alkylene)-(C₆-C₂₀ aryl). In some embodiments, the compound of formula (I) is a compound of formula (I-B), wherein R⁷ is optionally substituted —(C₁-C₂₀ alkylene)-(C₆-C₂₀ aryl)-(C₁-C₂₀ alkyl). In some embodiments, the compound of formula (I) is a compound of formula (I-B), wherein R⁷ is —(C₁-C₂₀ alkylene)-(C₆-C₂₀ aryl)-(C₁-C₂₀ alkyl). In some embodiments, the compound of formula (I) is a compound of formula (I-B), wherein R⁷ is unsubstituted —(C₁-C₂₀ alkylene)-(C₆-C₂₀ aryl)-(C₁-C₂₀ alkyl).

In some embodiments, the compound of formula (I) is a compound of formula (I-B), wherein R⁷ is optionally substituted phenyl. In some embodiments, the compound of formula (I) is a compound of formula (I-B), wherein R⁷ is phenyl. In some embodiments, the compound of formula (I) is a compound of formula (I-B), wherein R⁷ is unsubstituted phenyl.

In some embodiments, the compound of formula (I) is a compound of formula (I-C), wherein R⁷ is H. In some embodiments, the compound of formula (I) is a compound of formula (I-C), wherein R⁷ is optionally substituted C₁-C₂₀ alkyl. In some embodiments, the compound of formula (I) is a compound of formula (I-C), wherein R⁷ is C₁-C₂₀ alkyl. In some embodiments, the compound of formula (I) is a compound of formula (I-C), wherein R⁷ is unsubstituted C₁-C₂₀ alkyl. In some embodiments, the compound of formula (I) is a compound of formula (I-C), wherein R⁷ is optionally substituted C₆-C₂₀ aryl. In some embodiments, the compound of formula (I) is a compound of formula (I-C), wherein R⁷ is C₆-C₂₀ aryl. In some embodiments, the compound of formula (I) is a compound of formula (I-C), wherein R⁷ is unsubstituted C₆-C₂₀ aryl. In some embodiments, the compound of formula (I) is a compound of formula (I-C), wherein R⁷ is optionally substituted —(C₁-C₂₀ alkylene)-(C₆-C₂₀ aryl). In some embodiments, the compound of formula (I) is a compound of formula (I-C), wherein R⁷ is —(C₁-C₂₀ alkylene)-(C₆-C₂₀ aryl). In some embodiments, the compound of formula (I) is a compound of formula (I-C), wherein R⁷ is unsubstituted —(C₁-C₂₀ alkylene)-(C₆-C₂₀ aryl). In some embodiments, the compound of formula (I) is a compound of formula (I-C), wherein R⁷ is optionally substituted —(C₁-C₂₀ alkylene)-(C₆-C₂₀ aryl)-(C₁-C₂₀ alkyl). In some embodiments, the compound of formula (I) is a compound of formula (I-C), wherein R⁷ is —(C₁-C₂₀ alkylene)-(C₆-C₂₀ aryl)-(C₁-C₂₀ alkyl). In some embodiments, the compound of formula (I) is a compound of formula (I-C), wherein R⁷ is unsubstituted —(C₁-C₂₀ alkylene)-(C₆-C₂₀ aryl)-(C₁-C₂₀ alkyl).

In some embodiments, the compound of formula (I) is a compound of formula (I-C), wherein R⁷ is optionally substituted phenyl. In some embodiments, the compound of formula (I) is a compound of formula (I-C), wherein R⁷ is phenyl. In some embodiments, the compound of formula (I) is a compound of formula (I-C), wherein R⁷ is unsubstituted phenyl.

In some embodiments, the compound of formula (I) is selected from:

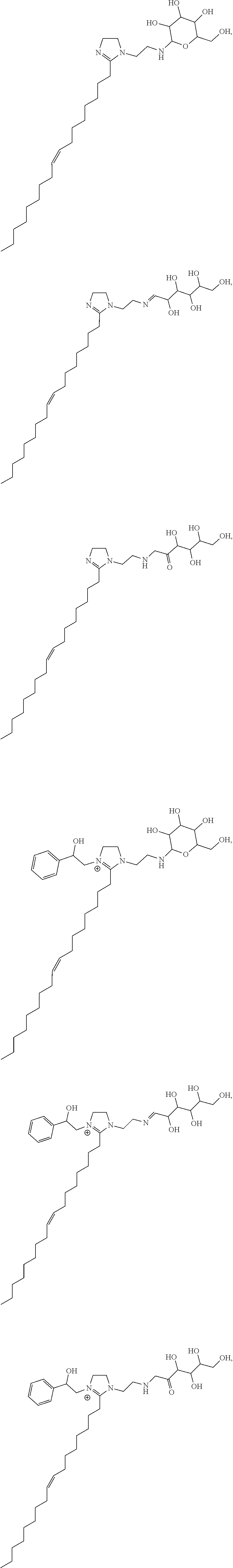 [SMILES: CCCCCCCC/C=C\CCCCCCCC1=NCCN1CCNC1OC(CO)C(O)C(O)C1O ; CCCCCCCC/C=C\CCCCCCCC1=NCCN1CC/N=C/C(O)C(O)C(O)C(O)CO ; CCCCCCCC/C=C\CCCCCCCC1=NCCN1CCNCC(=O)C(O)C(O)C(O)CO ; CCCCCCCC/C=C\CCCCCCCC1=[N+](CC(O)c2ccccc2)CCN1CCNC1OC(CO)C(O)C(O)C1O ; CCCCCCCC/C=C\CCCCCCCC1=[N+](CC(O)c2ccccc2)CCN1CC/N=C/C(O)C(O)C(O)C(O)CO ; CCCCCCCC/C=C\CCCCCCCC1=[N+](CC(O)c2ccccc2)CCN1CCNCC(=O)C(O)C(O)C(O)CO]

and salts thereof.

In some embodiments, the compound of formula (I) is:

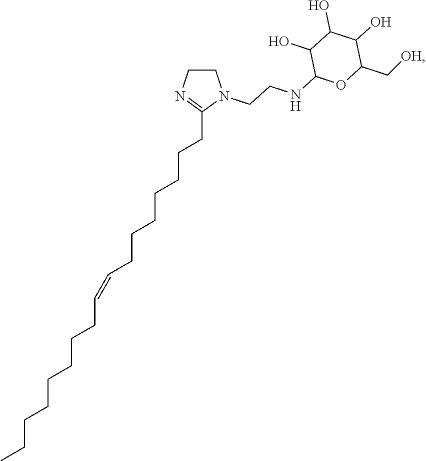 [SMILES: CCCCCCCC/C=C\CCCCCCCC1=NCCN1CCNC1OC(CO)C(O)C(O)C1O]

or a salt thereof.

In some embodiments, the compound of formula (I) is:

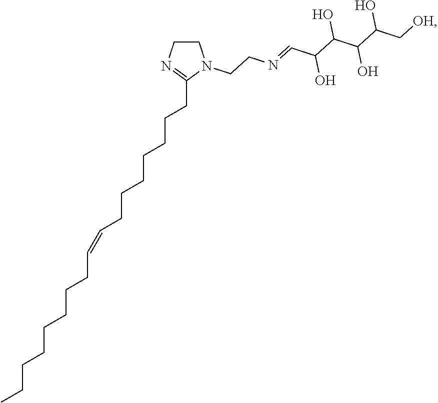 [SMILES: CCCCCCCC/C=C\CCCCCCCC1=NCCN1CC/N=C/C(O)C(O)C(O)C(O)CO]

or a salt thereof.

In some embodiments, the compound of formula (I) is:

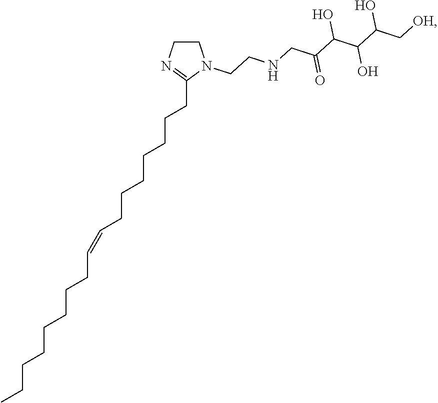 [SMILES: CCCCCCCC/C=C\CCCCCCCC1=NCCN1CCNCC(=O)C(O)C(O)C(O)CO]

or a salt thereof.

In some embodiments, the compound of formula (I) is:

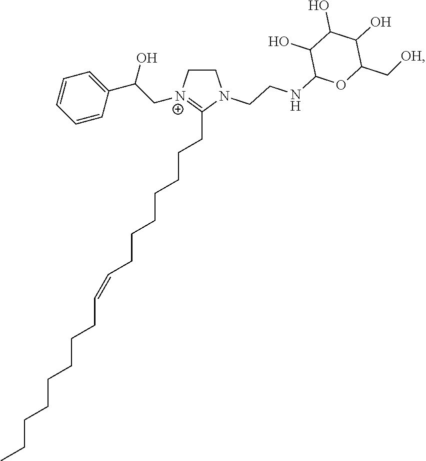 [SMILES: CCCCCCCC/C=C\CCCCCCCC1=[N+](CC(O)c2ccccc2)CCN1CCNC1OC(CO)C(O)C(O)C1O]

or a salt thereof.

In some embodiments, the compound of formula (I) is:

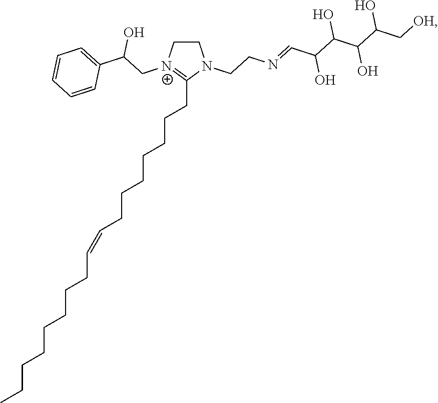 [SMILES: CCCCCCCC/C=C\CCCCCCCC1=[N+](CC(O)c2ccccc2)CCN1CC/N=C/C(O)C(O)C(O)C(O)CO]

or a salt thereof.

In some embodiments, the compound of formula (I) is:

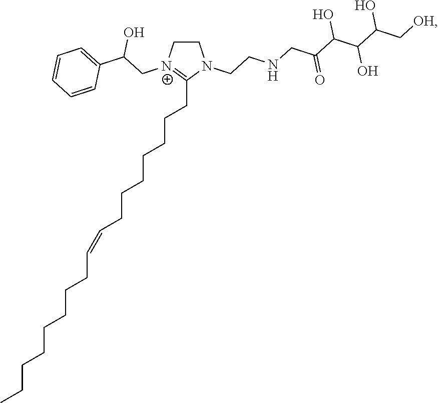 [SMILES: CCCCCCCC/C=C\CCCCCCCC1=[N+](CC(O)c2ccccc2)CCN1CCNCC(=O)C(O)C(O)C(O)CO]

or a salt thereof.

In some embodiments, the compound of formula (I) is provided in a salt form. In some embodiments, the composition of the disclosure includes the compound of formula (I) in a salt form. In some embodiments, the composition of the disclosure includes a salt of a compound of formula (I). In some embodiments, the salt of the compound of formula (I) is an acetate salt.

A method of reducing corrosion of a metal surface in contact with an aqueous system is also provided. In some aspects, the method includes adding a compound of formula (I), as described herein, to the aqueous system. In other aspects, the method includes adding a corrosion inhibitor composition that includes a compound of formula (I), as described herein, to the aqueous system.

The compositions and methods are capable of inhibiting corrosion of a metallic surface regardless of the type of corrodent in the aqueous medium. Typical corrodents include, for example, brine, an organic acid, carbon dioxide, and hydrogen sulfide.

In certain embodiments, the compositions and methods of the present disclosure are useful for inhibiting corrosion of metallic surfaces in contact with any type of corrodent in the medium, such as metal cations, metal complexes, metal chelates, organometallic complexes, aluminum ions, ammonium ions, barium ions, chromium ions, cobalt ions, cuprous ions, cupric ions, calcium ions, ferrous ions, ferric ions, hydrogen ions, magnesium ions, manganese ions, molybdenum ions, nickel ions, potassium ions, sodium ions, strontium ions, titanium ions, uranium ions, vanadium ions, zinc ions, bromide ions, carbonate ions, chlorate ions, chloride ions, chlorite ions, dithionate ions, fluoride ions, hypochlorite ions, iodide ions, nitrate ions, nitrite ions, oxide ions, perchlorate ions, peroxide ions, phosphate ions, phosphite ions, sulfate ions, sulfide ions, sulfite ions, hydrogen carbonate ions, hydrogen phosphate ions, hydrogen phosphite ions, hydrogen sulfate ions, hydrogen sulfite ions, an acid, such as carbonic acid, hydrochloric acid, nitric acid, sulfuric acid, nitrous acid, sulfurous acid, a peroxy acid, or phosphoric acid, ammonia, bromine, carbon dioxide, chlorine, chlorine dioxide, fluorine, hydrogen chloride, hydrogen sulfide, iodine, nitrogen dioxide, nitrogen monoxide, oxygen, ozone, sulfur dioxide, hydrogen peroxide, polysaccharides, metal oxides, sands, clays, silicon dioxide, titanium dioxide, muds, insoluble inorganic and/or organic particulates, an oxidizing agent, a chelating agent, an alcohol, and any combination of the foregoing.

The presently disclosed compositions and methods are useful for inhibiting corrosion of surfaces comprising any metal or combination of metals. In some aspects, the metal surface comprises steel, such as stainless steel or carbon steel. In some aspects, the metal surface comprises iron, aluminum, zinc, chromium, manganese, nickel, tungsten, molybdenum, titanium, vanadium, cobalt, niobium, or copper. The metal surface may also comprise any combination of the foregoing metals and/or any one or more of boron, phosphorus, sulfur, silicon, oxygen, and nitrogen. In some aspects, a pipe, heat exchanger, storage vessel, or a tank (e.g., railroad tank car or a tank truck/tanker) comprises the metallic surface.

In some aspects of the present disclosure, a metallic surface may comprise metallic-chrome steel, ferritic-alloy steel, austenitic-steel, precipitation-hardened steel, high-nickel steel, carbon steel, or a combination thereof.

While a compound of formula (I) or a salt thereof, as disclosed herein, can be added to an aqueous system at any dosage rate, illustrative examples of dosage rates include about 0.01 ppm to about 50,000 ppm. In other aspects, the compound of formula (I), or a salt thereof, is added to an aqueous system at a dosage rate of about 5 ppm to about 50,000 ppm. In other aspects, the compound of formula (I), or a salt thereof, is added to an aqueous system at a dosage rate of about 5 ppm to about 20,000 ppm. In other aspects, the compound of formula (I), or a salt thereof, is added to an aqueous system at a dosage rate of about 5 ppm to about 10,000 ppm. In other aspects, the compound of formula (I), or a salt thereof, is added to an aqueous system at a dosage rate of about 5 ppm to about 5,000 ppm. In other aspects, the compound of formula (I), or a salt thereof, is added to an aqueous system at a dosage rate of about 5 ppm to about 2,000 ppm. In other aspects, the compound of formula (I), or a salt thereof, is added to an aqueous system at a dosage rate of about 5 ppm to about 1,000 ppm. In other aspects, the compound of formula (I), or a salt thereof, is added to an aqueous system at a dosage rate of about 5 ppm to about 500 ppm. In other aspects, the compound of formula (I), or a salt thereof, is added to an aqueous system at a dosage rate of about 5 ppm to about 50 ppm. In other aspects, the compound of formula (I), or a salt thereof, is added to an aqueous system at a dosage rate of about 5 ppm to about 25 ppm. In other aspects, the compound of formula (I), or a salt thereof, is added to an aqueous system at a dosage rate of about 5 ppm to about 10 ppm.

In certain aspects, the compound of formula (I), or a salt thereof, is added to an aqueous system at a dosage rate of about 5 ppm. In certain aspects, the compound of formula (I), or a salt thereof, is added to an aqueous system at a dosage rate of about 10 ppm. In certain aspects, the compound of formula (I), or a salt thereof, is added to an aqueous system at a dosage rate of about 25 ppm. In certain aspects, the compound of formula (I), or a salt thereof, is added to an aqueous system at a dosage rate of about 50 ppm. In certain aspects, the compound of formula (I), or a salt thereof, is added to an aqueous system at a dosage rate of about 500 ppm. In certain aspects, the compound of formula (I), or a salt thereof, is added to an aqueous system at a dosage rate of about 1000 ppm. In certain aspects, the compound of formula (I), or a salt thereof, is added to an aqueous system at a dosage rate of about 2000 ppm. In certain aspects, the compound of formula (I), or a salt thereof, is added to an aqueous system at a dosage rate of about 5000 ppm. In certain aspects, the compound of formula (I), or a salt thereof, is added to an aqueous system at a dosage rate of about 10,000 ppm. In certain aspects, the compound of formula (I), or a salt thereof, is added to an aqueous system at a dosage rate of about 20,000 ppm. In certain aspects, the compound of formula (I), or a salt thereof, is added to an aqueous system at a dosage rate of about 50,000 ppm.

While a corrosion inhibitor composition and/or formulation as disclosed herein can be added to an aqueous system at any dosage rate, it may be added to an aqueous system at a dosage rate of from about 0.01 ppm to about 50,000 ppm. In other aspects, the corrosion inhibitor composition and/or formulation is added to an aqueous system at a dosage rate of about 5 ppm to about 50,000 ppm. In other aspects, the corrosion inhibitor composition and/or formulation is added to an aqueous system at a dosage rate of about 5 ppm to about 20,000 ppm. In other aspects, the corrosion inhibitor composition and/or formulation is added to an aqueous system at a dosage rate of about 5 ppm to about 10,000 ppm. In other aspects, the corrosion inhibitor composition and/or formulation is added to an aqueous system at a dosage rate of about 5 ppm to about 5,000 ppm. In other aspects, the corrosion inhibitor composition and/or formulation is added to an aqueous system at a dosage rate of about 5 ppm to about 2,000 ppm. In other aspects, the corrosion inhibitor composition and/or formulation is added to an aqueous system at a dosage rate of about 5 ppm to about 1,000 ppm. In other aspects, the corrosion inhibitor composition and/or formulation is added to an aqueous system at a dosage rate of about 5 ppm to about 500 ppm. In other aspects, the corrosion inhibitor composition and/or formulation is added to an aqueous system at a dosage rate of about 5 ppm to about 50 ppm. In other aspects, the corrosion inhibitor composition and/or formulation is added to an aqueous system at a dosage rate of about 5 ppm to about 25 ppm. In other aspects, the corrosion inhibitor composition and/or formulation is added to an aqueous system at a dosage rate of about 5 ppm to about 10 ppm.

In certain aspects, the corrosion inhibitor composition and/or formulation is added to an aqueous system at a dosage rate of about 5 ppm. In certain aspects, the corrosion inhibitor composition and/or formulation is added to an aqueous system at a dosage rate of about 10 ppm. In certain aspects, the corrosion inhibitor composition and/or formulation is added to an aqueous system at a dosage rate of about 25 ppm. In certain aspects, the corrosion inhibitor composition and/or formulation is added to an aqueous system at a dosage rate of about 50 ppm. In certain aspects, the corrosion inhibitor composition and/or formulation is added to an aqueous system at a dosage rate of about 500 ppm. In certain aspects, the corrosion inhibitor composition and/or formulation is added to an aqueous system at a dosage rate of about 1000 ppm. In certain aspects, the corrosion inhibitor composition and/or formulation is added to an aqueous system at a dosage rate of about 2000 ppm. In certain aspects, the corrosion inhibitor composition and/or formulation is added to an aqueous system at a dosage rate of about 5000 ppm. In certain aspects, the corrosion inhibitor composition and/or formulation is added to an aqueous system at a dosage rate of about 10,000 ppm. In certain aspects, the corrosion inhibitor composition and/or formulation is added to an aqueous system at a dosage rate of about 20,000 ppm. In certain aspects, the corrosion inhibitor composition and/or formulation is added to an aqueous system at a dosage rate of about 50,000 ppm.

The metal corrosion rate provided by a corrosion inhibitor composition and/or formulation as disclosed herein is not limited. In certain embodiments, a corrosion inhibitor composition and/or formulation as disclosed herein provides a metal corrosion rate that is acceptable according to industry standards, e.g., about 0.2 mpy or less. In certain aspects, a corrosion inhibitor composition and/or formulation as disclosed herein provides a metal corrosion rate of about 0.1 mpy or less. In additional aspects, a corrosion inhibitor composition and/or formulation as disclosed herein provides a metal corrosion rate of about 0.1 mpy or less, about 0.05 mpy or less, about 0.04 mpy or less, about 0.03 mpy or less, about 0.02 mpy or less, about 0.01 mpy or less, about 0.005 mpy or less, or about 0.002 mpy or less.

The corrosion inhibitor compositions and/or formulations as disclosed herein can be used to inhibit corrosion of metal in an aqueous system having any pH. In certain aspects, a corrosion inhibitor composition and/or formulation as disclosed herein is added to an aqueous system having a pH of from about 1 to about 12, from about 1 to about 11, from about 1 to about 10, from about 1 to about 9, from about 1 to about 8, from about 1 to about 7, from about 2 to about 12, from about 3 to about 12, from about 4 to about 12, from about 2 to about 10, or from about 3 to about 10, for example.

In certain aspects, a corrosion inhibitor composition and/or formulation as disclosed herein inhibits metal corrosion when added to an aqueous system comprising a biocide, such as a non-halogen-containing oxidizing biocide including, but not limited to, peroxides (e.g., hydrogen peroxide), persulfates, permanganates, and peracetic acids.

The corrosion inhibitor compositions and/or formulations as disclosed herein may be applied to the aqueous mediums and/or metallic surfaces disclosed herein by any suitable method. In certain embodiments, a corrosion inhibitor composition (or solution comprising the composition) and/or formulation as disclosed herein is applied by immersion, spraying, or other coating techniques. In certain embodiments, a corrosion inhibitor composition and/or formulation is introduced into the aqueous medium by any conventional method, such as manually or automatically using a chemical injection pump, and is fed into the aqueous system on either a periodic or continuous basis.

In certain aspects, if a corrosion inhibitor composition and/or formulation as disclosed herein is relatively insoluble in water, the composition may be made soluble by forming an organic or inorganic salt of one or more of the compounds within the composition/formulation. Thus, in certain aspects, a corrosion inhibitor composition and/or formulation as disclosed herein comprises a water-soluble salt of one or more of the compounds disclosed herein. In certain aspects, a corrosion inhibitor composition and/or formulation as disclosed herein is added as a solution in a water-miscible co-solvent including, but not limited to, acetone, methanol, ethanol, propanol, formic acid, formamide, propylene glycol, or ethylene glycol. In certain embodiments, a co-solvent is used to achieve maximum solubility of a corrosion inhibitor composition and/or formulation as disclosed herein in the aqueous system. In certain aspects, low molecular weight polyethylene glycol, polypropylene glycol, a surfactant (e.g., organic sulfonic acid), or combinations thereof are used to increase the solubility of a corrosion inhibitor composition and/or formulation as disclosed herein.

Those skilled in the art will appreciate that the corrosion inhibitor compositions and/or formulations disclosed herein can be added to an aqueous system alone or in combination with other corrosion inhibitors or treatment chemicals. Multiple corrosion inhibitors can be dosed as a combined corrosion inhibitor formulation or each corrosion inhibitor can be added separately, including two or more corrosion inhibitor compositions as disclosed herein. Moreover, the corrosion inhibitor compositions and/or formulations disclosed herein can be added to an aqueous system in combination with a variety of additional corrosion inhibitors including, but not limited to, azoles, orthophosphate, polyphosphates, phosphonates, molybdates, silicates, oximes, and nitrites.

The corrosion inhibitor compositions and/or formulations disclosed herein also can be added to an aqueous system before, after, and/or with an additional component selected from a fouling control agent, an additional corrosion inhibitor, a corrosion inhibitor intensifier, a biocide, a preservative, an acid, a hydrogen sulfide scavenger, a surfactant, an asphaltene inhibitor, a paraffin inhibitor, a scale inhibitor, a gas hydrate inhibitor, a pH modifier, an emulsion breaker, a reverse emulsion breaker, a coagulant/flocculant agent, an emulsifier, a water clarifier, a dispersant, an antioxidant, a polymer degradation prevention agent, a permeability modifier, a foaming agent, an antifoaming agent, a CO₂ scavenger, an O₂ scavenger, a gelling agent, a lubricant, a friction reducing agent, a salt, a clay stabilizer, a bactericide, a salt substitute, a relative permeability modifier, a breaker, a fluid loss control additive, a chelating agent, a demulsifier, an iron control agent, a drag reducing agent, a flow improver, a viscosity reducer, a solvent, and any combination thereof.

Illustrative examples of scale inhibitors include allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, pantoic acid, a salt thereof, or any combination thereof.

The corrosion inhibitor composition may comprise various amounts of each component. For example, in some embodiments, the composition may comprise from about 99% by weight water and about 1% by weight of one or more corrosion inhibitor compounds of formula (I) to about 50% by weight water and 50% by weight of one or more corrosion inhibitor compounds of formula (I). In other embodiments, the composition may comprise from about 99% by weight water and about 1% by weight of one or more corrosion inhibitor compounds of formula (I). In other embodiments, the composition may comprise from about 95% by weight water and about 5% by weight of one or more corrosion inhibitor compounds of formula (I). In other embodiments, the composition may comprise from about 90% by weight water and about 10% by weight of one or more corrosion inhibitor compounds of formula (I). In other embodiments, the composition may comprise from about 85% by weight water and about 15% by weight of one or more corrosion inhibitor compounds of formula (I). In other embodiments, the composition may comprise from about 80% by weight water and about 20% by weight of one or more corrosion inhibitor compounds of formula (I). In other embodiments, the composition may comprise from about 75% by weight water and about 25% by weight of one or more corrosion inhibitor compounds of formula (I). In other embodiments, the composition may comprise from about 70% by weight water and about 30% by weight of one or more corrosion inhibitor compounds of formula (I). In other embodiments, the composition may comprise from about 65% by weight water and about 35% by weight of one or more corrosion inhibitor compounds of formula (I). In other embodiments, the composition may comprise from about 60% by weight water and about 40% by weight of one or more corrosion inhibitor compounds of formula (I). In other embodiments, the composition may comprise from about 55% by weight water and about 45% by weight of one or more corrosion inhibitor compounds of formula (I). In other embodiments, the composition may comprise from about 50% by weight water and about 50% by weight of one or more corrosion inhibitor compounds of formula (I).

The corrosion inhibitor compositions and/or formulations as disclosed herein can be added to an aqueous system in any form. In certain aspects, a corrosion inhibitor composition and/or formulation is added to an aqueous system as a dried solid. In certain embodiments, a corrosion inhibitor composition and/or formulation is added to an aqueous system as a solution in a co-solvent miscible with water. In certain embodiments, a corrosion inhibitor composition and/or formulation as disclosed herein is added to an aqueous system as an aqueous solution.

In certain aspects, a corrosion inhibitor composition and/or formulation as disclosed herein is added to an oil or natural gas recovery system, an oil or natural gas transportation system, an oil or natural gas storage system, an oil or natural gas processing system, an aqueous system that recirculates water, and/or an aqueous system that has stagnant water.

A process for preparing a compound of formula (I) is additionally provided. The process for preparing a compound of formula (I) includes contacting a compound of formula (II):

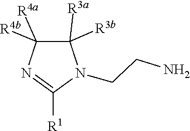

with a carbohydrate, wherein:
Y is N or N⁺—R⁵;
R¹ is an optionally substituted C₅-C₂₀ alkyl, an optionally substituted C₈-C₂₀ alkenyl, an optionally substituted C₅-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl);
R² is a carbohydrate group;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R⁵ is H, optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl).

In some aspects, the compound of formula (II) is contacted with the carbohydrate in the presence of an acid, such as an inorganic acid. One of ordinary skill in the art will appreciate that there are many such acids that are compatible with the process and would be capable of selecting such an acid without the burden of undue experimentation. In some aspects, the acid is an organic acid. In some aspects, the acid is a sulfonic acid, such as p-toluenesulfonic acid, a phosphonic acid, and/or a carboxylic acid, such as acetic acid.

In some aspects, the compound of formula (II) is contacted with the carbohydrate in the presence of heat. One of ordinary skill in the art will appreciate that there are many reaction temperatures compatible with the process and would be capable of selecting such a reaction temperature without the burden of undue experimentation. In some aspects, the compound of formula (II) is contacted with the carbohydrate at a temperature in the range of about 70° C. to about 150° C. In other aspects, the compound of formula (II) is contacted with the carbohydrate at a temperature the range of about 75° C. to about 145° C. In other aspects, the compound of formula (II) is contacted with the carbohydrate at a temperature the range of about 80° C. to about 140° C. In other aspects, the compound of formula (II) is contacted with the carbohydrate at a temperature the range of about 85° C. to about 140° C. In other aspects, the compound of formula (II) is contacted with the carbohydrate at a temperature the range of about 90° C. to about 135° C. In other aspects, the compound of formula (II) is contacted with the carbohydrate at a temperature the range of about 90° C. to about 130° C. In other aspects, the compound of formula (II) is contacted with the carbohydrate at a temperature the range of about 90° C. to about 125° C. In other aspects, the compound of formula (II) is contacted with the carbohydrate at a temperature the range of about 90° C. to about 120° C. In other aspects, the compound of formula (II) is contacted with the carbohydrate at a temperature the range of about 90° C. to about 115° C. In other aspects, the compound of formula (II) is contacted with the carbohydrate at a temperature the range of about 90° C. to about 110° C. In other aspects, the compound of formula (II) is contacted with the carbohydrate at a temperature the range of about 90° C. to about 105° C. In other aspects, the compound of formula (II) is contacted with the carbohydrate at a temperature the range of about 90° C. to about 100° C. In other aspects, the compound of formula (II) is contacted with the carbohydrate at a temperature of about 95° C. In other aspects, the compound of formula (II) is contacted with the carbohydrate under reflux at a temperature of about 95° C.

In some aspects, the process for preparing a compound of formula (I) further includes contacting a compound of formula (III):

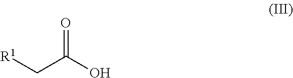

with a compound of formula (IV):

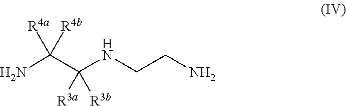

to produce the compound of formula (II).

In some aspects, the process for preparing a compound of formula (I) includes contacting the compound of formula (II) with a carbohydrate, wherein the carbohydrate is a reducing sugar.

In some aspects, the process for preparing a compound of formula (I) includes contacting the compound of formula (II) with a carbohydrate, wherein the carbohydrate is a reducing monosaccharide or a reducing disaccharide. In other aspects, the process for preparing a compound of formula (I) includes contacting the compound of formula (II) with a carbohydrate, wherein the carbohydrate is a reducing monosaccharide. In other aspects, the process for preparing a compound of formula (I) includes contacting the compound of formula (II) with a carbohydrate, wherein the carbohydrate is a reducing disaccharide.

In some aspects, the process for preparing a compound of formula (I) includes contacting the compound of formula (II) with a carbohydrate, wherein the carbohydrate is selected from glyceraldehyde, erythrose, threose, arabinose, lyxose, ribose, xylose, allose, altrose, glucose, mannose, gulose, idose, galactose, talose, fructose, sedoheptulose, mannoheptulose, lactose, maltose, sucrose, or any combination thereof.

### EXAMPLES

The foregoing may be better understood by reference to the following examples, which are intended for illustrative purposes and are not intended to limit the scope of the disclosure or its application in any way.

### Example 1. Synthesis of (Z)-2-((2-(2-(heptadec-8-en-1-yl)-4,5-dihydro-1H-imidazol-1-yl)ethyl)amino)-6-(hydroxymethyl)tetrahydro-2H-pyran-3,4,5-triol

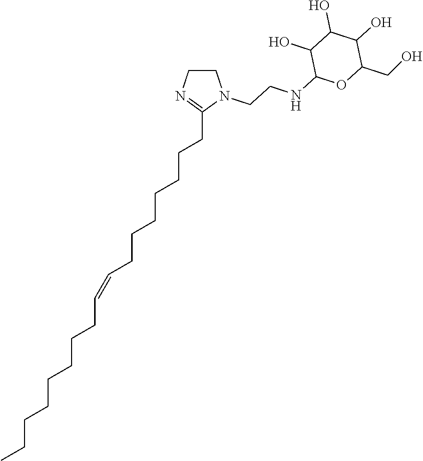 [SMILES: CCCCCCCC/C=C\CCCCCCCC1=NCCN1CCNC1OC(CO)C(O)C(O)C1O]

An imidazoline was synthesized using oleic acid (OA) and diethylenetriamine (DETA) according to methods commonly known to those of ordinary skill in the art. The OA/DETA imidazoline was used as the imidazoline prototype for modification with a carbohydrate.

Along with a magnetic follower and 200 g of deionized water, the imidazoline, 51.687 g (146.84 mmol.), was added into a three-neck 1-L round-bottomed flask. The flask was armed with a thermocouple and a water-cooled condenser. Following the vigorous agitation of the reaction mixture in a reactor on a heating mantle, 26.454 g (146.84 mmol.) of anhydrous glucose was charged into the reaction mixture. A solution of 100 g of deionized water and 3.246 g (16.81 mmoles; 11% mol. eq.) of p-toluene sulfonic acid hydrate was added to the reactor. The reaction mixture was refluxed at 95° C.

Within 30 minutes, the reaction mixture turned from a light amber slurry to a deep brown slurry. After 90 minutes, the reaction mixture was cooled to room temperature for recovery of the title compound. On cooling to 25° C., solvent was removed in vacuo to yield a gum-textured product, used to make prototype compositions Formula 1 and Formula 2.

### Example 2. Synthesis of (Z)-2-((2-(2-(heptadec-8-en-1-yl)-4,5-dihydro-1H-imidazol-1-yl)ethyl)amino)-6-(hydroxymethyl)tetrahydro-2H-pyran-3,4,5-triol acetate salt (Formula 1)

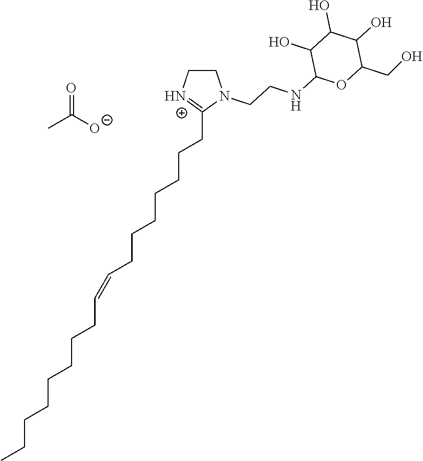

As in Example 1, the OA/DETA imidazoline (51.687 g: 146.84 mmol.) was used as a prototype for modification with a carbohydrate. The OA/DETA imidazoline was added into a reactor followed by a magnetic follower, 100 g of ethanol and 100 g of deionized water. Glacial acetic acid (8.48 mL: 146.84 mmol.) was charged into the reaction mixture dropwise. The mixture was vigorously agitated until the solution was homogenous. To the mixture was added 26.454 g (146.84 mmol.) of anhydrous glucose. The reaction mixture was refluxed at 95° C. After 60 minutes, the reaction mixture was cooled to room temperature for recovery of the acetate salt, Formula 1.

### Example 3. Synthesis of (Z)-2-(heptadec-8-en-1-yl)-3-(2-hydroxy-2-phenylethyl)-1-(2-((3,4,5-trihydroxy-6-(hydroxymethyl)tetrahydro-2H-pyran-2-yl)amino)ethyl)-4,5-dihydro-1H-imidazol-3-ium acetate salt (Formula 2)

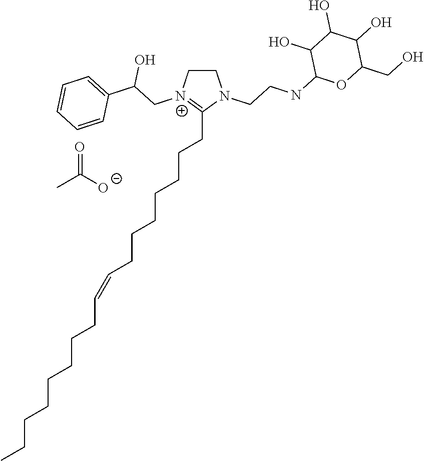

Into a reactor containing crude product made via the method described in Example 1, and used without the removal of the water solvent, 8.48 mL (146.84 mmol.) of glacial acetic acid was added in a dropwise fashion. The mixture was stirred until all the clumps were dissolved under reflux at 95° C. Using a dropping funnel, 17.10 mL (146.84 mmol.) of styrene oxide was added dropwise to the reaction mixture. The mixture was refluxed for 2 hours at 95° C., after which it was cooled to room temperature to yield Formulation 2 upon dilution to 481.4 mM.

### Example 4. Blank Wheel Box Test

To determine the efficacy of the compositions as corrosion inhibitors, a wheel box test was used. It is a testing methodology from NACE publication ID182. The method is used to assess the degree of protection afforded to a metal surface that is in contact with water laden with carbon dioxide (C₀₂). The wheel box test is a test that is often used in the field for comparative corrosion inhibitor performance tests. The standard test conditions used in the performance test are in Table 1.

TABLE 1
Wheel Box Test Conditions

| Temperature | 80° | C. |
|---|---|---|
| Oil type | 20% LVT-200 | |
| Brine¹ | 80% ASTM Seawater brine | |
| CO₂ level | Saturated | |
| Duration | 24 | hours |

¹The brine solution consisted of 0.47 g/L of CaCl₂•2H₂O, 0.60 g/L of MgCl₂—6H₂0, 0.11 g/L of KCl, 0.08 g/L of Na₂SO₄, and 1.4 g/L of NaHCO₃.

A continuous wheel box test procedure was used to compare the performance of the corrosion inhibitor formulations of the invention in mitigating the corrosion of carbon steel in solutions saturated with carbon dioxide. Into a set of bottles containing the desired brine and oil mixtures, Table 1, clean and pre-weighed coupons were immersed. The solution was purged with carbon dioxide, after which the solutions were dosed with the various concentrations of the formulations. After purging the headspace with carbon dioxide, the bottles were immediately capped to prevent aerial exposure.

Blanks, comprising bottles of untreated mixtures, were used to determine the baseline corrosion rate under the test conditions. The bottles were placed on a rotating wheel under incubation at 80° C. for a period of 24 hours. Following the completion of the incubation, the bottles were retrieved and left to cooled to room temperature. The coupons were removed from the test solutions, cleaned, dried, sandblasted and re-weighed. By comparing the mass loss of the coupons immersed in treated solutions versus the mass loss for coupons placed in untreated solutions, the corrosion rate and protection efficiency were calculated.

TABLE 2
Blank Corrosion Test (in triplicate)

| | | Weight | | |
|---|---|---|---|---|
| | Inhibitor Conc. | Loss | | % |
| | (ppm) | Mg. | MPY | Protection |
| BLANK | 0 | 66.6 | 40.63 | 0% |
| | 0 | 68.3 | 41.66 | 0% |
| | 0 | 64 | 39.04 | 0% |

The data in Table 2 demonstrate that the test coupons were not protected in the presence of untreated test solutions.

### Example 5. Comparative Formulation Wheel Box Test

To determine the efficacy of the compositions as corrosion inhibitors, the method in Example 4 was used. The inhibitor composition was prepared using 418.4 mM solution of OA-DETA imidazolium acetate:

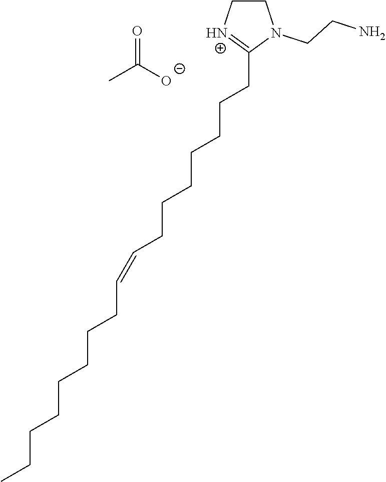

in water. According to the test, this composition afforded an average protection efficiency of percentage of 89%, Table 3.

TABLE 3
Corrosion Test for Comparative Formulation

| | Inhibitor | | | |
|---|---|---|---|---|
| | Conc. | Weight | | % |
| | (ppm) | Loss Mg. | MPY | Protection |
| Comparative | 500 | 7.7 | 4.7 | 88% |
| Formulation | 500 | 7 | 4.27 | 89% |
| | 1000 | 6.6 | 4.03 | 90% |
| | 1000 | 7.2 | 4.39 | 89% |
| | 2000 | 6.9 | 4.21 | 90% |
| | 2000 | 7.1 | 4.33 | 89% |
| | 5000 | 7.6 | 4.64 | 89% |
| | 5000 | 7 | 4.27 | 89% |
| | 10000 | 7.3 | 4.45 | 89% |
| | 10000 | 6.9 | 4.21 | 90% |
| | 20000 | 7.4 | 4.51 | 89% |
| | 20000 | 7.2 | 4.39 | 89% |
| | 50000 | 6.9 | 4.21 | 90% |
| | 50000 | 6.7 | 4.09 | 90% |

Moreover, the protection was robust even at low treatment doses. However, the formulation was physically unstable. The OA-DETA imidazolium acetate salt flocculated out of solution, which is highly undesirable, as solids in a product are known to plug transfer lines and quills.

### Example 6. Novel Formulation 2 Wheel Box Test

The method of Example 4 was used to determine the efficacy of the novel composition, Formula 1. The sample of Formula 1 used in this test was prepared by the methods of Example 2. The formulation contained 418.4 mM of (Z)-2-((2-(2-(heptadec-8-en-1-yl)-4,5-dihydro-1H-imidazol-1-yl)ethyl)amino)-6-(hydroxymethyl)tetrahydro-2H-pyran-3,4,5-triol acetate salt.

TABLE 4
Corrosion Test for Formula 2

| | Inhibitor | Weight | | |
|---|---|---|---|---|
| | Conc. (ppm) | Loss Mg. | MPY | % Protection |
| Formulation 1 | 500 | 3.4 | 2.07 | 95% |
| | 500 | 3.3 | 2.01 | 95% |
| | 1000 | 4.4 | 2.68 | 93% |
| | 1000 | 4.2 | 2.56 | 94% |
| | 2000 | 4.6 | 2.81 | 93% |
| | 2000 | 3.7 | 2.26 | 94% |
| | 5000 | 4 | 2.44 | 94% |
| | 5000 | 3.9 | 2.38 | 94% |
| | 10000 | 3.6 | 2.2 | 95% |
| | 10000 | 3.7 | 2.26 | 94% |
| | 20000 | 2.9 | 1.77 | 96% |
| | 20000 | 3 | 1.83 | 95% |
| | 50000 | 3.1 | 1.89 | 95% |
| | 50000 | 4 | 2.44 | 94% |

According to the data presented in Table 4, Formula 2 displayed greatly improved corrosion inhibitor performance even at low dosages. The inhibitor also displayed high solubility in water, unlike the comparative sample of oleic acid-DETA imidazolium acetate salt (Example 5).

### Example 7. Novel Formulation 3 Wheel Box Test

The method of Example 4 was used to determine the corrosion inhibitor efficacy of 418.4 mM of Formula 2, as synthesized in Example 3.

TABLE 5
Corrosion Test for Formula 3

| | Inhibitor | Weight Loss | | % |
|---|---|---|---|---|
| | Conc. (ppm) | Mg. | MPY | Protection |
| Formulation 2 | 500 | 5.7 | 3.48 | 91% |
| | 500 | 5.2 | 3.17 | 92% |
| | 1000 | 5.3 | 3.23 | 92% |
| | 1000 | 5.5 | 3.36 | 92% |
| | 2000 | 5.8 | 3.54 | 91% |
| | 2000 | 5.1 | 3.11 | 92% |
| | 5000 | 5.3 | 3.23 | 92% |
| | 5000 | 4.9 | 2.99 | 93% |
| | 10000 | 4.4 | 2.68 | 93% |
| | 10000 | 3.8 | 2.32 | 94% |
| | 20000 | 3.8 | 2.32 | 94% |
| | 20000 | 4.0 | 2.44 | 94% |
| | 50000 | 4.1 | 2.50 | 94% |
| | 50000 | 4.0 | 2.44 | 94% |

According to the data presented in Table 5, Formula 2 was highly effective. Moreover, the acetate salt was physically stable.

### Example 8. Low Dosage Comparison of Comparative Formulation, Formula 1, and Formula 2

The method of Example 4 was used to determine the corrosion inhibitor efficacy of the Comparative Formulation, Formula 1, and Formula 2 at lower dosage rates.

TABLE 6
Low Dosage Comparison

| | | | % Protection by (Z)-2- |
|---|---|---|---|
| | | | (heptadec-8-en-1-yl)-3- |
| | % Protection by | % Protection by (Z)-2- | (2-hydroxy-2- |
| | (Z)-2-(heptadec- | (heptadec-8-en-1-yl)-1- | phenylethyl)-1-(2- |
| | 8-en-1-yl)-1-(2- | (2-((3,4,5,6- | ((3,4,5,6- |
| | hydroxyethyl)- | tetrahydroxytetrahydro- | tetrahydroxytetrahydro- |
| | 4,5-dihydro-1H- | 2H-pyran-2- | 2H-pyran-2- |
| | imidazol-3-ium | yl)amino)ethyl)-4,5- | yl)amino)ethyl)-4,5- |
| ppm of | Acetate | dihydro-1H-imidazol-3- | dihydro-1H-imidazol-3- |
| Corrosion | (Comparative | ium Acetate | ium Acetate |
| Inhibitor | Formulation) | (Formula 1) | (Formula 2) |
| 5 | 14 | 22 | 28 |
| | | (54% improvement) | (100% improvement) |
| 10 | 24 | 32 | 36 |
| | | (33% improvement) | (50% improvement) |
| 25 | 42 | 54 | 57 |
| | | (29% improvement) | (36% improvement) |
| 50 | 55 | 63 | 73 |
| | | (15% improvement) | (33 improvement) |

The data presented in Table 6 demonstrate that Formula 1 and Formula 2 perform significantly better than the comparative sample even at low dosage rates.

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a corrosion inhibitor” is intended to include “at least one corrosion inhibitor” or “one or more corrosion inhibitors.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

What is claimed is:

1. A corrosion inhibitor composition comprising a compound of formula (I): 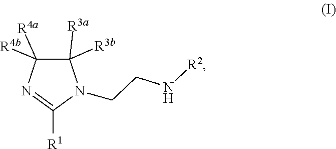
or a salt thereof, wherein:
Y is N or N⁺—R⁵;
R¹ is an optionally substituted C₈-C₂₀ alkyl functional group, an optionally substituted C₈-C₂₀ alkenyl with one or more vinylic moieties, an optionally substituted C₈-C₂₀ alkynyl with one or more vinylic moieties, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl);
R² is a carbohydrate group;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R⁵ is H, optionally substituted C₁-C₂₀ alkyl, optionally substituted C₂-C₂₀ β-hydroxyalkyl, C₂-C₂₀β-hydroxy-β-phenylalkyl, optionally substituted —(C₁-C₂₀ alkylene)-(C₆-C₂₀ aryl), optionally substituted —(C₂-C₂₀ β-hydroxyalkylene)-(C₆-C₂₀ aryl), or optionally substituted —(C₂-C₂₀ β-hydroxyalkylene)-(C₆-C₂₀ aryl)-(C₆-C₂₀ alkyl).

2. The composition of claim 1, wherein Y is N or N⁺—R⁵.

3. The composition of claim 1, wherein R¹ is selected from octyl, octanyl, octynyl, nonyl, nonenyl, nonynyl, decyl, decenyl, decynyl, undecyl, undecenyl, undecynyl, dodecyl, dodecenyl, dodecynyl, tridecyl, tridecenyl, tridecynyl, tetradecyl, tetradecenyl, tetradecynyl, pentadecyl, pentadecenyl, pentadecynyl, hexadecyl, hexadecenyl, hexadecynyl, heptadecyl, heptadecenyl, heptadecynyl, octadecyl, octadecenyl, octadecynyl, nonadecyl, nonadecenyl, nonadecynyl, icosyl, icosenyl, and icosynyl.

4. The composition of claim 1, wherein R^{3a} is H, wherein R^{4a} is H, wherein R^{4b} is H, and/or wherein R⁵ is —CH₂CH(OH)-phenyl, —CH₂CH₂(OH)—CH₂CH(OH)-alkyl, or —CH₂CH(OH)-phenylalkyl.

5. The composition of claim 1, wherein R² is a reducing sugar group, optionally wherein R² is a reducing monosaccharide or reducing disaccharide.

6. The composition of claim 1, wherein R² is a Maillard adduct of a reducing sugar group or an Amadori adduct of a reducing sugar group.

7. The composition of claim 1, wherein R² is selected from glyceraldehyde, erythrose, threose, arabinose, lyxose, ribose, xylose, allose, altrose, glucose, mannose, gulose, idose, galactose, talose, fructose, sedoheptulose, mannoheptulose, lactose, maltose, and sucrose.

8. The composition of claim 1, wherein the compound of formula (I) is a compound of formula (I-A), (I-B), or (I-C): 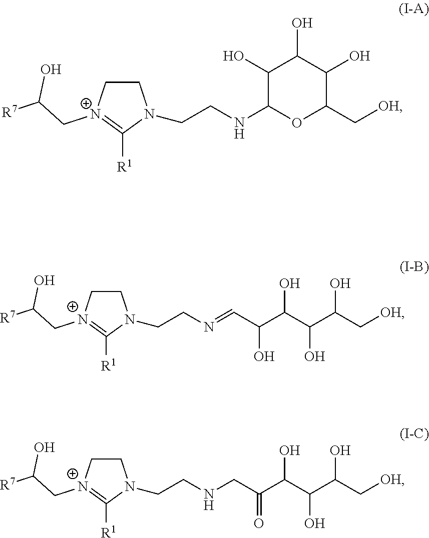
or a salt thereof, wherein:
R¹ is an optionally substituted C₅-C₂₀ alkyl, an optionally substituted C₈-C₂₀ alkenyl, an optionally substituted C₅-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl); and
R⁷ is H, optionally substituted C₁-C₂₀ alkyl, C₆-C₂₀ aryl, optionally substituted —(C₁-C₂₀ alkylene)-(C₆-C₂₀ aryl), or optionally substituted —(C₁-C₂₀ alkylene)-(C₆-C₂₀ aryl)-(C₁-C₂₀ alkyl).

9. The composition of claim 1, comprising a compound of formula (I) selected from: 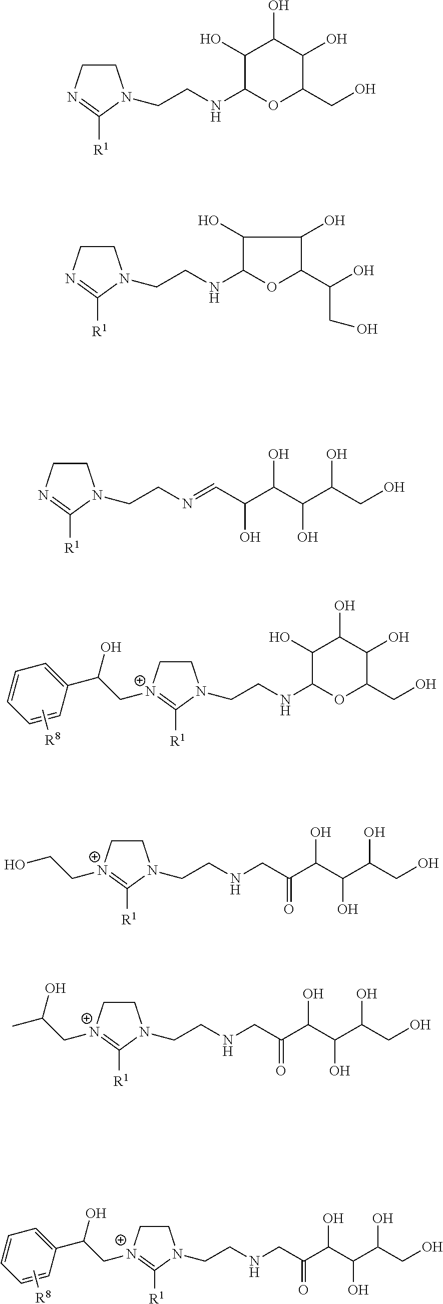 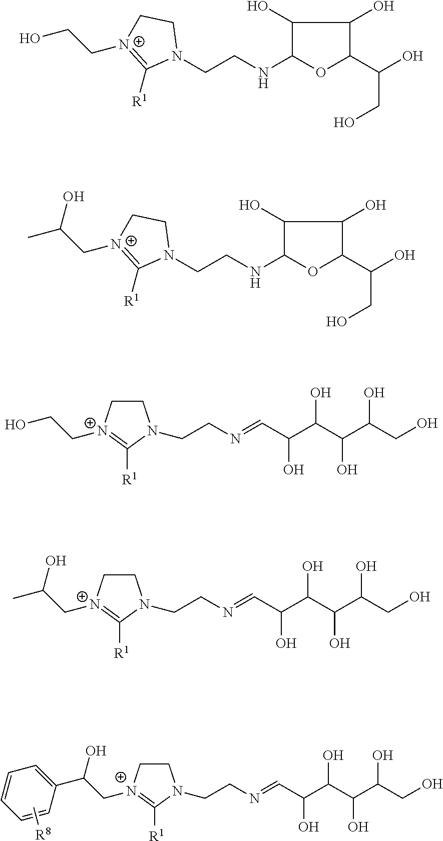 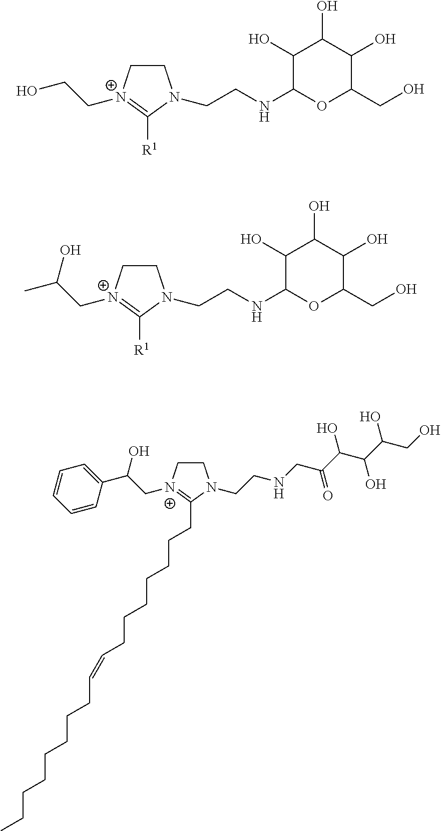
and salts thereof, wherein R¹ is an optionally substituted C₈-C₂₀ alkyl, an optionally substituted C₈-C₂₀ alkenyl, an optionally substituted C₈-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl); and
wherein R⁸ is an alkyl, an aryl, an alkyl aryl or aryl alkyl group.

10. The composition of claim 1, further comprising a salt of a compound of formula (I).

11. The composition of claim 1, further comprising an antiscalant selected from the group consisting of allaric acid, altaric acid, altraric acid, altronic acid, arabinaric acid, arabinonic acid, citric acid, dihomocitric acid, fructuronic acid, fuconic acid, fumaric acid, galactaric acid, galactonic acid, galacturonic acid, glucaric acid, glucoheptonic acid, gluconic acid, glucuronic acid, gulonic acid, homocitric acid, homoisocitric acid, idaric acid, idonic acid, iduronic acid, isocitric acid, mannaric acid, mannonic acid, malic acid, tartaric acid, octulosonic acid, rhamnonic acid, ribonic acid, tagaturonic acid, xylonic acid, xyluronic acid, tartaric acid, tatronic acid, glyceric acid, malonic acid, pantoic acid, a salt thereof, and any combination thereof.

12. A method of reducing corrosion of a metal surface in contact with an aqueous system, comprising adding the corrosion inhibitor composition of claim 1 to the aqueous system.

13. The method of claim 12, wherein the aqueous system comprises a corrosive agent selected from a brine, an organic acid, carbon dioxide, hydrogen sulfide, or any combination thereof.

14. The method of claim 12, wherein the metal surface comprises metallic-chrome steel, ferritic-alloy steel, austenitic-steel, precipitation-hardened steel, high-nickel steel, carbon steel, or a combination thereof.

15. A process for preparing a compound of formula (I): 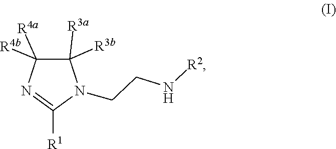
comprising contacting a compound of formula (II): 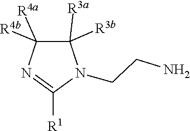
with a carbohydrate,
wherein:
Y is N or N⁺—R⁵;
R¹ is an optionally substituted C₈-C₂₀ alkyl, an optionally substituted C₈-C₂₀ alkenyl, an optionally substituted C₈-C₂₀ alkynyl, an optionally substituted —(C₆-C₂₀ aryl)-(C₄-C₂₀ alkyl), an optionally substituted —(C₄-C₂₀ alkylene)-(C₆-C₂₀ aryl), or an optionally substituted —(C₄-C₂₀ alkylene)-(C₆-C₂₀ aryl)-(C₄-C₂₀ alkyl);
R² is a carbohydrate group;
R^{3a} is H, —OH, —NH₂, —NH(C₁-C₆ alkyl), —N(C₁-C₆ alkyl)₂, —CN, halo, or optionally substituted C₁-C₆ alkyl;
R^{3b} is H, —OH, —NH₂, —NH(C₁-C₆ alkyl), —N(C₁-C₆ alkyl)₂, —CN, halo, or optionally substituted C₁-C₆ alkyl;
R^{4a} is H, —OH, —NH₂, —NH(C₁-C₆ alkyl), —N(C₁-C₆ alkyl)₂, —CN, halo, or optionally substituted C₁-C₆ alkyl;
R^{4b} is H, —OH, —NH₂, —NH(C₁-C₆ alkyl), —N(C₁-C₆ alkyl)₂, —CN, halo, or optionally substituted C₁-C₆ alkyl; and
R⁵ is H, optionally substituted C₁-C₂₀ alkyl, optionally substituted C₂-C₂₀ hydroxyalkyl, optionally substituted —(C₁-C₂₀ alkylene)-(C₆-C₂₀ aryl), optionally substituted —(C₂-C₂₀ hydroxyalkylene)-(C₆-C₂₀ aryl), or optionally substituted —(C₂-C₂₀ hydroxyalkylene)-(C₆-C₂₀ aryl)-(C₆-C₂₀ alkyl).

16. The process of claim 15, wherein contacting a compound of formula (II) with a carbohydrate is performed in the presence of an acid and/or heat.

17. The process of claim 15, further comprising contacting a compound of formula (III): 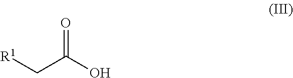
with a compound of formula (IV): 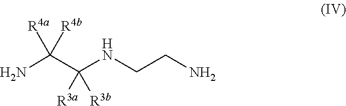
to produce the compound of formula (II).

18. The process of claim 15, wherein the carbohydrate is a reducing sugar.

19. The process of claim 15, wherein the carbohydrate is selected from glyceraldehyde, erythrose, threose, arabinose, lyxose, ribose, xylose, allose, altrose, glucose, mannose, gulose, idose, galactose, talose, fructose, sedoheptulose, mannoheptulose, lactose, maltose, and sucrose.
